(19) United States
(12) Patent Application Publication
(10) Pub. No.: US 2017/0157858 A1
(43) Pub. Date: Jun. 8, 2017

(54) METHOD OF MANUFACTURING BLOCK TYPE SCAFFOLD

(71) Applicant: THE CATHOLIC UNIVERSITY OF KOREA INDUSTRY-ACADEMIC COOPERATION FOUNDATION, Seoul (KR)

(72) Inventor: Sang Hwa LEE, Seoul (KR)

(21) Appl. No.: 15/123,183

(22) PCT Filed: Jul. 7, 2016
(86) PCT No.: PCT/KR2016/007349

§ 371 Date: Sep. 1, 2016

(30) Foreign Application Priority Data
Jul. 22, 2015 (KR) 10-2015-0103940

(51) Int. Cl. B29C 67/00; B33Y 70/00; B33Y 80/00; A61F 2/28; B33Y 50/02

(52) U.S. Cl. CPC B29C 67/0088; A61F 2/2803; A61F 2/28; B33Y 50/02; B33Y 80/00; B33Y 70/00; A61F 2002/2835; A61F 2240/002; B29K 2067/00

(57) ABSTRACT

Provided is a technique of manufacturing a scaffold for jaw bone or bone reconstruction, by which a scaffold is fast and accurately manufactured to be placed instantly after treatment and simultaneously a scaffold is manufactured to be stably placed in an irregular recipient site or a wide region such as a tumor. A method of manufacturing a block type scaffold include generating a three-dimensional (3D) image of a scaffold to be placed in a recipient site for bone reconstruction by using a medical image file (DICOM file) of CT image data, dividing the 3D image of the scaffold into a plurality of block images, and manufacturing the scaffold by outputting the 3D image of the scaffold by using a 3D printer, wherein a plurality of 3D objects corresponding to the plurality of block images are output to be separable from one another.

### TECHNICAL FIELD

The present disclosure relates to a technique of manufacturing a scaffold to help treatment of jaw bone or bone reconstruction, and more particularly, to a technique of accurately manufacturing a scaffold in a reconstruction area and simultaneously manufacturing a scaffold to be stably placed in an irregular recipient site or a wide region such as a tumor.

### BACKGROUND ART

Bone reconstruction of an affected part such as a jawbone in the field of dentistry has been considered as an essential part for rapid restoration through improvement of a treatment effect. In particular, for a wide affected part generated due to a tumor, bone reconstruction is difficult and a long treatment time is needed. Accordingly, various technologies to improve a treatment effect have been researched.

Various scaffolds have been manufactured and used for treatments of jawbones or bone reconstruction. For example, in the field of dentistry, Korean Patent No. 10-1527934 discloses a technology of manufacturing a scaffold for dental treatment by using various materials. Related arts disclose technical properties of a dental implant or scaffold having osseointegration and osteogenesis effects with respect to scaffolds for dental treatment and capable of emitting compositions for promoting osteogenesis.

However, the above related arts present only a configuration, such as a detailed material, of a scaffold. Furthermore, there has been no research for a technology to substantially accurately place a scaffold in a recipient site.

### DETAILED DESCRIPTION OF THE INVENTION

### Technical Problem

The present invention provides a technique of manufacturing a scaffold for jawbone or bone reconstruction in a block shape to accurately match the shape of an affected part, prior to treatment, by which a scaffold to be instantly placed and simultaneously stably placed in a wide region such as a tumor or in an irregular recipient site may be manufactured. Also, in the manufacturing of various blocks, various materials for assisting osteogenesis are added to each block so that blocks may be evenly and widely added to an implantation area.

### Technical Solution

According to a first embodiment of the present invention, there is provided a method of manufacturing a block type scaffold, which includes generating a three-dimensional (3D) image of a scaffold to be placed in a recipient site for bone reconstruction by using a medical image file (DICOM file) of CT image data, dividing the 3D image of the scaffold into a plurality of block images, and manufacturing the scaffold by outputting the 3D image of the scaffold by using a 3D printer, wherein a plurality of 3D objects corresponding to the plurality of block images are output to be separable from one another.

### Advantageous Effects of the Invention

According to embodiments, a scaffold to be placed in a recipient site may be manufactured by accurately anticipating an image of the scaffold by using a medical image file included in computed tomography (CT) image data scanned before treatment. In particular, a scaffold may be manufactured by placing and combining a plurality of blocks in a wide area or in an area in which a scaffold may not be stably placed to match the shape of the area as a large space is formed inside due to generation of undercut.

Accordingly, a scaffold that is instantly placed during actual treatment may be manufactured so that a fast and accurate treatment is possible. In particular, a scaffold may be manufactured to be placed accurately corresponding to a recipient site having a shape that varies according to treatments. Furthermore, a scaffold that is stably placed in a wide region such as a tumor or in an irregular recipient site may be manufactured so that a treatment effect may be much increased.

### DESCRIPTION OF THE DRAWINGS

FIG. 1 is a flowchart of a method of manufacturing a block type scaffold according to an embodiment.

FIG. 2 is an image for explaining an example of placing a block type scaffold, according to an embodiment.

### BEST MODE

Hereinafter, a method of manufacturing a block type scaffold according to an embodiment of the present disclosure will now be described with reference to accompanying drawings.

For the purposes of promoting an understanding of the principles of the inventive concept, well-known functions or constructions are not described in detail. It should be understood that embodiments described herein should be considered in a descriptive sense only and not for purposes of limitation. Descriptions of features or aspects within each embodiment should typically be considered as available for other similar features or aspects in other embodiments.

Throughout the specification, like reference numerals refer to like elements throughout and redundant descriptions thereof are not provided here.

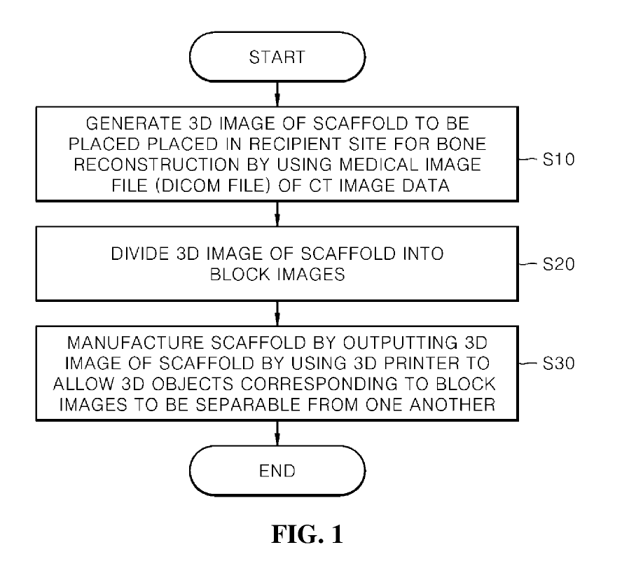

FIG. 1 is a flowchart of a method of manufacturing a block type scaffold according to an embodiment.

Referring to FIG. 1, a method of manufacturing a block type scaffold according to the present embodiment includes generating a three-dimensional (3D) image of a scaffold to be placed in a recipient site (or in an affected part) for bone reconstruction by using a medical image file (Digital Imaging and Communications in Medicine (DICOM) file) of computed tomography (CT) image data (S10).

CT is also referred to as cone beam computed tomography (CBCT) that is a scanner capable of scanning the inside of an object to be scanned and making the object three-dimensionally recognizable. When a CT image is captured by using a CT imaging apparatus, CT image data is generated and the data includes the medical image file (DICOM file) corresponding to a 3D image of the inside of the object.

For example, in the case of dental CT, the medical image file generated as a result of CT imaging includes images of alveolar bone, teeth, lost parts, and tumor, which are distinguishable from one another. A recipient site (jawbone, a tooth extraction socket during tooth extraction, and other bone loss part) as a position where an expected scaffold is to be placed after treatment according to a state of a patient may be determined by using the medical image file.

A 3D image of a scaffold to be placed in a recipient site for bone reconstruction may be generated by using the properties of a medical image file, in which alveolar bone and teeth are distinguishable, in the operation S10.

When a 3D image of a scaffold is generated in the operation S10, the 3D image of a scaffold is divided into a plurality of block images (S20).

In the operation S20, the dividing of the 3D image of a scaffold into a plurality of block images signifies dividing the 3D image of a scaffold into a plurality of objects. The block images may vary according to the shape of a scaffold and the shape of a recipient site as a placement position.

For example, when a large scaffold is placed in a recipient site that is wide and easy to place a scaffold, the blocks may have various shapes. When an affected part is located inside a human body, which has a wide inner space while having a narrow external appearance, such as an undercut, so that a scaffold is difficult to be inserted into the affected part and a 3D image of a scaffold for a part corresponding to the undercut may be divided into a plurality of block images.

When the block images are formed, the scaffold is manufactured to be divided into a plurality of blocks according to the above-described block images. The manufactured scaffold blocks may be stably coupled to one another.

In other words, when the scaffold image is divided into the block images, the block images may be generated to have a coupling structure to be coupled with adjacent 3D objects, for each 3D object, such that the 3D objects included in the scaffold and manufactured corresponding to the block images are separated from one another and also fixed to one another when coupled to one another.

For example, for each block image, the block image is generated to have a structure to be forcibly coupled to the adjacent block images so that stably fixing between the 3D objects may be available when a scaffold is manufactured and placed later.

When the operation S20 is performed, the 3D image of a scaffold is output by using a 3D printer. The 3D objects corresponding to the block images generated in the operation

S20 are output to be capable of being separated from one another as described above, thereby manufacturing a scaffold (S30).

In the operation S30, in detail, a variety of methods may be used, for example, a method of outputting a scaffold, by one-time output, in a state in which a plurality of block images are coupled to one another but to be separable from one another or a method of separately manufacturing a plurality of 3D objects included in the scaffold by outputting using a 3D printer for each 3D object corresponding to the block images.

Since the scaffold is manufactured by using a strip including bio-affinitive polymer (e.g., polycaprolactone (PCL)) for a 3D printing output, it is possible to manufacture a bio-affinitive scaffold and to increase an effect on treatment.

In detail, during the manufacture of a scaffold, to further improve the bone reconstruction effect by using the properties of the 3D objects corresponding to the block images, a strip including a cell corresponding to the recipient site and a growth factor of the cell for a 3D printing output may be used so that the cell and the growth factor are included in the center of each of the 3D objects corresponding to the block images. Accordingly, the cell and the growth factor are included in the central portions of the 3D objects and thus the treatment effect may be further improved.

According to the above flow, a scaffold may be manufactured by analyzing the shape of a part subject to the bone reconstruction including alveolar bone and teeth of a patient by using the medical image file and anticipating a treatment result. In particular, since a scaffold that is stably placed in a wide region such as a tumor or in an irregular recipient site may be manufactured, the treatment effect may be much improved. The above may be applied to an affected part for bone reconstruction in addition to the above-described alveolar bone and teeth.

After the scaffold is manufactured in the above process, an additional operation of placing the scaffold in a recipient site of an actual patient may be performed. When the scaffold is placed, a method of sequentially placing and fixing a plurality of 3D objects may be used.

Accordingly, even when an actual treatment on a patient is not performed, not a scaffold having a uniform shape, but a scaffold that may be accurately placed in a recipient site generated when an actual treatment on a patient is performed may be manufactured regardless of whether a treatment is performed or not, and thus a scaffold may be instantly placed during the actual treatment on a patient. Also, since a scaffold may be manufactured with high accuracy to be accurately placed in an actual recipient site, the treatment effect may be further improved through the stable fixing of the scaffold in the recipient site.

In particular, since the scaffold is manufactured to be divided into the 3D objects and also to be capable of being coupled to one another, while a scaffold to be placed in a wide region such as a tumor may be manufactured and stably placed, a scaffold to be placed in the recipient site, for example, an undercut, where a scaffold that is integrally manufactured may not be inserted, may be manufactured so that the scaffold is stably placed in the recipient site by sequentially placing and assembling 3D objects in the recipient site.

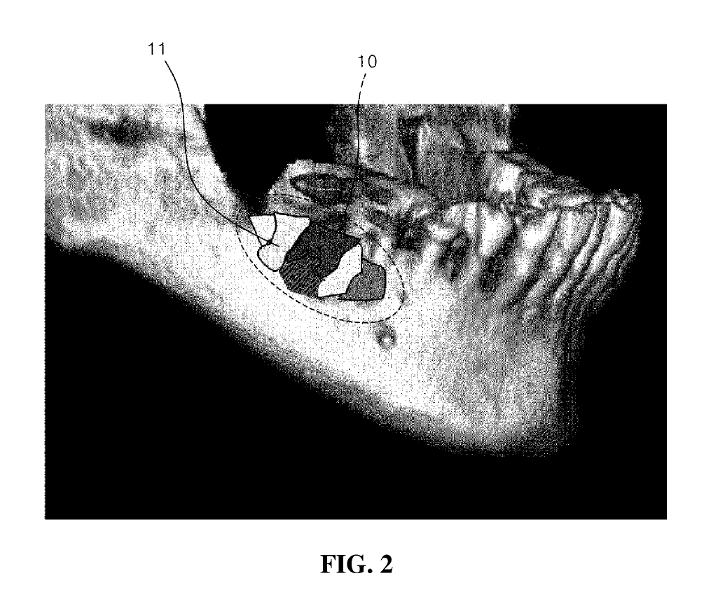

FIG. 2 is an image for explaining an example of placing a block type scaffold, according to an embodiment.

Referring to FIG. 2, in a medical image file, a region 10 where a 3D image of a scaffold is generated may be checked. In other words, the shape of the region 10 such as a bone state in the region 10 is analyzed and then a 3D image of a scaffold to be placed after treatment is generated.

According to the present embodiment the 3D image of a scaffold may be divided into a plurality of block images 11. Although FIG. 2 illustrates the block images 11 in two dimensions for convenience of explanation, the block images 11 may have shapes in 3D.

It will be apparent that all elements of the one or more embodiments of the present inventive concept are not limited to be combined or to operate as one combination. That is, all elements may be selectively combined and may operate as one within the scope of the present inventive concept.

In addition, when a part “includes”, “comprises”, “is configured of”, or “has” an element, unless there is a particular description contrary thereto, the part can further include other elements, not excluding the other elements. Unless expressly described otherwise, all terms including descriptive or technical terms which are used herein should be construed as having meanings that are obvious to one of ordinary skill in the art. Also, terms that are defined in a general dictionary and that are used in the following description should be construed as having meanings that are equivalent to meanings used in the related description, and unless expressly described otherwise herein, the terms should not be construed as being ideal or excessively formal.

While the present inventive concept has been particularly shown and described with reference to exemplary embodiments thereof, it will be understood by one of ordinary skill in the art that various changes in form and details may be made therein without departing from the spirit and scope of the following claims. Therefore, the scope of the present inventive concept is defined not by the detailed description of the present inventive concept but by the appended claims, and all differences within the scope will be construed as being included in the present inventive concept. CLAIMS

1. A method of manufacturing a block type scaffold, the method comprising:
generating a three-dimensional (3D) image of a scaffold to be placed in a recipient site for bone reconstruction by using a medical image file (DICOM file) of CT image data;
dividing the 3D image of the scaffold into a plurality of block images; and
manufacturing the scaffold by outputting the 3D image of the scaffold by using a 3D printer, wherein a plurality of 3D objects corresponding to the plurality of block images are output to be separable from one another.

2. The method of claim 1, wherein, in the manufacturing of the scaffold, the scaffold is manufactured by using a strip including bio-affinitive material for a 3D printing output.

3. The method of claim 2, wherein, in the manufacturing of the scaffold, the scaffold is manufactured by using a strip including a cell corresponding to the recipient site and a growth factor of the cell for a 3D printing output so that the cell and the growth factor are included in each of the 3D objects corresponding to the plurality of block images

4. The method of claim 1, wherein, in the dividing of the 3D image of the scaffold into the plurality of block images, the plurality of block images are formed to have a coupling structure to be coupled with adjacent 3D objects, for each of the plurality of 3D objects, such that the plurality of 3D objects are separated from one another and also fixed to one another when coupled to one another.

5. The method of claim 4, wherein the coupling structure is a forcibly insertion structure.
